(19) United States
(12) Patent Application Publication
(10) Pub. No.: US 2017/0000949 A1
(43) Pub. Date: Jan. 5, 2017

(54) MECHANISMS FOR INJECTION AND DOSING

(71) Applicant: Allergan, Inc., Irvine, CA (US)

(72) Inventors: Ethan Franklin, Santa Barbara, CA (US); Zachary Dominguez, Santa Barbara, CA (US)

(21) Appl. No.: 15/179,466

(22) Filed: Jun. 10, 2016

Related U.S. Application Data
(60) Provisional application No. 62/186,995, filed on Jun. 30, 2015.

(51) Int. Cl. A61M 5/315

(52) U.S. Cl. CPC A61M 5/3156; A61M 5/31555

(57) ABSTRACT

A mechanically operable handpiece is provided which allows a physician to easily deliver repeated, incremental doses of a beneficial composition, for example, a dermal filler gel, superficially into skin.

### CROSS REFERENCE TO RELATED APPLICATIONS

This application claims the benefit of U.S. Provisional Patent Application No. 62/186,995 filed Jun. 30, 2015, the entire disclosure of which is incorporated herein by this specific reference.

The present invention generally relates to mechanisms for injection and dosing, and more specifically relates to devices for providing minute doses of pharmaceuticals, therapeutic compositions, dermal filler compositions and the like, superficially into skin.

### BACKGROUND

Aesthetic dermal filler procedures have become increasing popular in recent years, as they have proven to be quite effective in improving the appearance of the face, for example, in reducing the signs of aging by smoothing wrinkles and folds, such as the nasolabial folds, and plumping the midface. Some of the more popular dermal fillers are soft, colorless injectable gel compositions made of hyaluronic acid. Hyaluronic acid (HA) is a long chain polymer, more specifically, a polysaccharide, that is naturally occurring in body tissues. When chemically crosslinked, hyaluronic acid makes an excellent, long lasting, dermal filler material. Dermal filler procedures generally involve injections of dermal filler materials into the skin or subdermally, commonly in an area of a wrinkle, depression or volume loss, for example. The procedures are quite minimally invasive, and the results are nearly immediate, and may last for several months to a year or even longer. Hyaluronic acid naturally and harmlessly degrades in the body tissues, and thus the results are ultimately temporary. Furthermore, in the event of unsatisfactory results, such as overfill, the injected filler can be removed quickly using hyaluronidase.

Conventional dermal filler procedures are generally performed by injection of the composition into or below the skin using a standard syringe and a fine gauge needle. A typical dermal filler patient may undergo from about 5 to about 10 injections in a single procedure, with injection points across various regions of the face. While the goal may be to improve the appearance of the entire face, a skilled aesthetic physician generally aims to correct one or more specific regions of the face, for example, regions that lack volume such as the lips or the cheeks, or regions that present specific wrinkles, such as deep nasolabial folds, with specific input from the patient regarding areas he or she finds detracting to his or her appearance.

It has been discovered that improvement of facial appearance can also be accomplished by introducing minute amounts of compositions into skin at a very superficial depth, and across wide regions of the skin, rather than focusing on specific wrinkles or specific areas of the face that lack volume.

What is needed is a device for facilitating multiple, controlled, minute dosing of dermal fillers, or any other therapeutic, aesthetically enhancing or pharmaceutical composition, into the skin as a desired depth.

### SUMMARY

The present invention is generally directed to a device that can be used to deliver a composition, for example a dermal filler gel, into skin to improve the appearance of the skin, and perhaps improve overall skin health and quality. In some embodiments, the device facilitates treatment of a large surface area of skin, such as the entire face, neck and/or décolletage.

The device allows for enhanced controlled depth of injection, especially for superficial delivery of compositions, for example, dermal filler gels.

Advantageously, the device is more efficient at delivering doses of dermal filler to a large surface area of skin than is currently possible with a standard needle and syringe.

In one embodiment, a device is provided for delivering repeated doses of a dermal filler superficially into skin, the device generally comprising a handpiece comprising a main housing having a housing distal end; a cartridge containing a dermal filler and having a cartridge distal end configured to be coupled to a needle; a plunger movable within the cartridge, and having a distal portion defining a head and a proximal portion defining a drive rack; a shuttle, movable within the main housing and structured to receive the cartridge; and retention clips for holding the shuttle forward in the housing and drive clips engaging the drive rack; the device being structured and configured to allow one-way, incremental movement of the plunger each time the device is device actuated, thereby allowing delivery of repeated doses of the dermal filler into skin. In some embodiments, the device is structured to be actuated upon pressing the device against the skin, which moves the shuttle back (proximally) relative to the housing, overcoming a bias of the retention clips to hold the shuttle forward. In some embodiments, the drive rack comprises spaced apart elements, such as teeth, leaves, projections, notches or other features that engage the drive clips, and the incremental movement of the plunger is defined by spacing between these adjacent features, e.g. teeth, of the drive rack.

In other embodiments, the device further comprises a trigger which actuates the device, to cause incremental, repeatable injections of dermal filler upon pressing the trigger.

These and other features and advantages of the invention may be better appreciated and understood by referring to the following Detailed Description and accompanying Drawings, which show certain non-limiting, exemplary embodiments of the invention.

### BRIEF DESCRIPTION OF THE DRAWINGS

FIGS. 1-4 show, in cross-sectional view, working components of a handpiece of a device in accordance with one embodiment of the invention.

FIGS. 5-14 show, in cross-sectional view, working components of another handpiece of a device in accordance with another embodiment of the invention.

### DETAILED DESCRIPTION

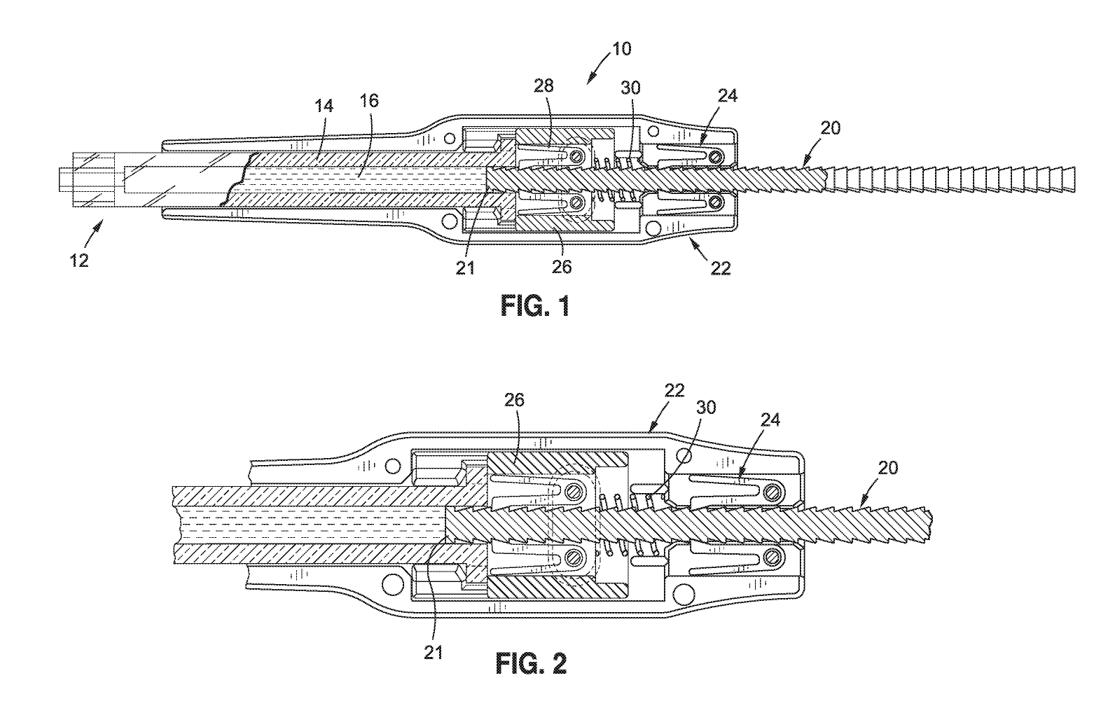

An exemplary embodiment of the invention is shown, in simplified view, in cross-section in FIG. 1. A distal-most end (to the left side of drawings) of the device 10 includes a coupling end for connecting to a needle (not shown) for delivering a dermal filler composition. For purposes of the present disclosure, the device will hereinafter generally be described as being used to inject dermal filler, for example, a hyaluronic acid-based dermal filler gel, but it is to be understood that the device can also be used, within the scope of the invention, to inject other compositions that would be aesthetically or therapeutically beneficial when injected into the skin or beneath the skin.

More specifically, device 10 includes a distal coupling end 12 that is structured to be couplable to a needle, or alternatively, to a plurality of needles (not shown). The needle may extend beyond a distal most surface of the device approximately between about 0.5 mm and about 5.0 mm, for example, between about 1.0 mm and about 3.0 mm depending on the depth the filler is to be delivered into skin.

Device 10 may comprise a syringe cartridge 14 filled with product (e.g. dermal filler gel 16, a drive rack 20 having one way teeth features, and plunger 21 within the cartridge 14. Device 10 further includes an outer housing 22, drive clips 24 coupled to the housing 22, an inner shuttle 26 including retention clips 28, a spring 30 which forces the shuttle 26 forward within the housing 22, and several dowel pins. Syringe cartridge 14 is fixed to, and moves with, the shuttle 26.

To use device 10 for delivering multiple, repeated, small doses of composition (hereinafter, for the sake of simplicity, the composition will be referred to as a dermal filler) into skin, a user grasps the device 10 by the outer housing 22. The distal end 12 of the device 10 is placed against the skin to be treated. The act of placing the device against the skin of the patient applies pressure to the front of the syringe 14, which drives the shuttle 26 backward, and drives the outer housing forward, which in turn delivers a dose of the dermal filler gel out of a needle (not shown) secured to the distal coupling end 12 of the syringe.

More specifically, in the shown embodiment, two sets of one-way clips incrementally move the plunger 21, via the drive rack 20, forward each time the device 10 is actuated or pressed against the skin and then released. The amount of dose delivered is related to the spacing or pitch of the teeth on the drive rack 20.

In its rest position, shown in FIG. 2, the shuttle 26 is forward (distal) in the housing 22 (forced into that position by the spring 30). Drive clips 24 are engaged in the rack 20.

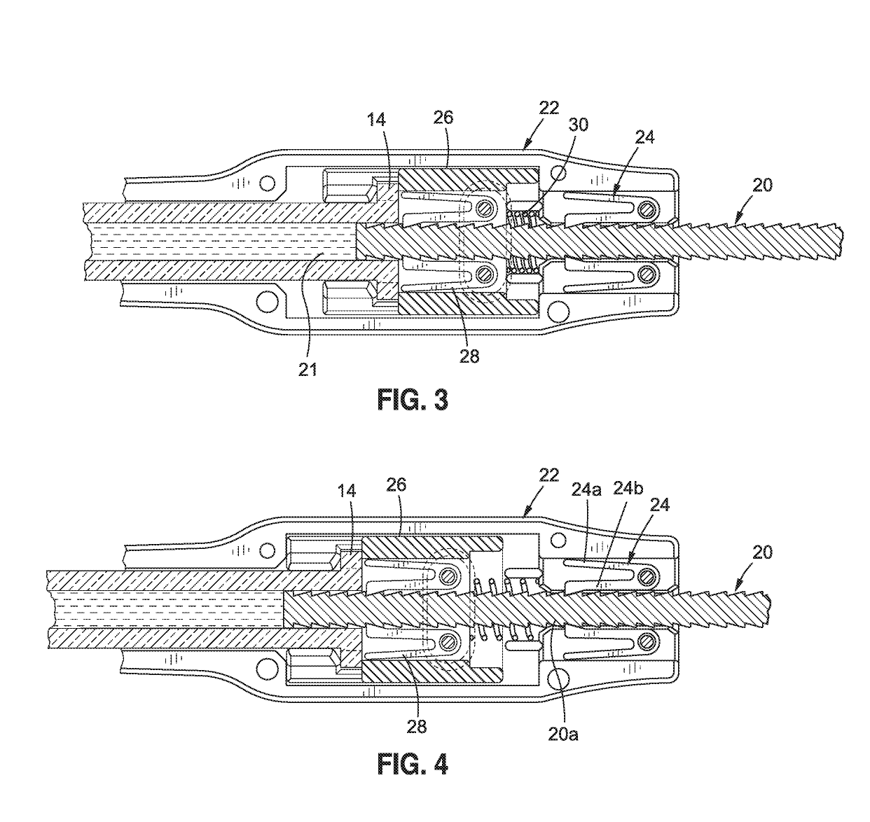

To use, the syringe distal end is placed into contact with the skin until the needle (not shown) penetrates the skin. As shown in FIG. 3, as pressure is applied to the outer housing by the user, the outer housing 22 is moved forward. Simultaneously, the shuttle 26 (which is fixed to syringe) moves backward, compressing the spring 30. Note that because the syringe 14 is attached to the shuttle 26, the syringe 14 also moves backward relative to the outer housing 22. Because the drive clips 24 are engaged in the rack 20, the rack 20 is held in position relative to the outer housing 22 and is moved forward into the syringe 14 relative to the syringe 14/shuttle 26. Distal (forward) end of rack 20 functions as a plunger in syringe 14, which pushes out an incremental dose of the composition from syringe 14 and into the skin. The retention clips 28 are overridden during this stage.

As shown in FIG. 4, as the user releases forward pressure from the outer housing 22, the syringe 14/shuttle 26 moves forward (left) due to the spring force. Due to the retention clips 28 (left), the rack is held in its new position relative to the syringe 14/shuttle 26 and the drive clips 24 (right) are overridden. When the shuttle 26 is in its full forward position (as shown), the drive clips 24 engage into the next set of teeth on the drive rack 20 (right), ready for the subsequent actuation and dosing.

Note that this mechanism provides both dosing and needle puncture against skin, as well as accurate dose control. An effect of this mechanism is that to the user it feels as though these two steps (needle puncture and dosing) are completed in one smooth, rapid and precise movement. The delivered dose amount is driven by the pitch of the drive rack, as well as the displacement of the shuttle within the outer housing. Another notable feature of this design is the clips themselves. Each clip may be identical and potentially easily molded (e.g. straight pull mold). Each clip, for example clip 24 shown in FIG. 4, may comprise a first leg 24a and an opposing second leg 24b. First leg 24a acts or functions as a spring against a housing feature, while second leg 24b engages tooth 20a of drive rack 20. Also, note that because the pivot point of each clip is off-axis from the contact point of the clip tooth with the gear rack, an over-center effect is created, which effectively “locks” the clips into place under any reverse loading scenarios, adding to the strength of the clip mechanism.

In one embodiment, the depth at which the device 10 can deliver composition into skin is between about 500 μm and about 2000 μm. When used to deliver a hyaluronic acid based dermal filler gel, this depth will place the gel into or just beneath the superficial dermis, and may provide beneficial skin quality improvement. In other embodiments, the depth at which the device delivers composition into skin is greater than 2000 μm, for example, may be between about 1 mm and about 10 mm, for example, about 2 mm, about 3 mm, about 4 mm, about 5 mm, about 6 mm, about 7 mm, about 8 mm, about 9 mm, about 10 mm, or greater. Further, in some embodiments, the device allows for controlled dosing.

In some embodiments, the device is structured to be capable of providing controlled dosing of doses of about 5 μL to about 100 μL with each injection. Other embodiments may provide doses of less than about 5 μL. Other embodiments may provide doses of greater than about 100 μL, for example, about 200 μL, about 300 μL, about 400 μL, about 500 μL, about 600 μL, about 700 μL, about 800 μL, about 900 μL about 1000 μL, or more.

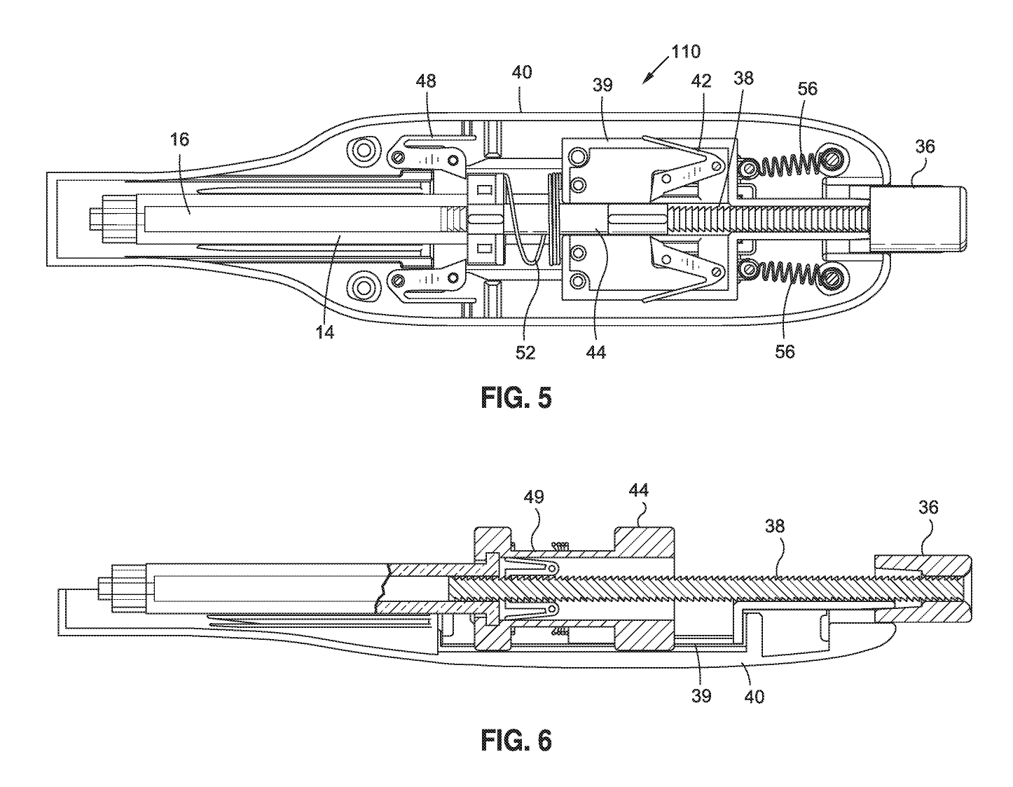

In another embodiment shown in FIGS. 5-14, a device 110 is provided which is similar to the device 10 shown in FIGS. 1-4, in that it provides dosing in a similar way. However, this device 110 is actuated independently from the placement against the skin.

In this embodiment, the device 110 is actuated by a button 36 on the rear (right side in Figures) of the device 110. However, one can easily transfer the basics of this mechanism to an alternative drive system, e.g. a manually actuated lever, a solenoid, a DC gear motor, a stepper motor, etc.

Advantageously, device 110 includes a mechanism that will snap the cartridge 14 and cause needle or needles (not shown) to penetrate into and retract from the skin (the distal end of the device is toward the left in these Figures). The present structure allows for a repeatable penetration scenario, which may result in more repeatable, consistent doses at consistent depths. This shown embodiment comprises, for example, syringe cartridge 14 filled with composition or product 16 (e.g. hyaluronic acid based gel), a drive rack 38 with one way teeth features, an outer housing 40, a button/drive mechanism subassembly 39 which includes a trigger or button, and driving clips 42. A shuttle 44 holds the cartridge 14 and contains shuttle retention clips 49 (cross-section FIG. 6). Device 110 further includes shuttle retention clips 48, compression spring 52 located between the button/drive mechanism assembly 39 and the shuttle 44. A pair of extension springs 56 are provided for connecting the button/drive mechanism to the housing 40.

A cross sectional view of device 110 is shown in FIG. 6. Note the retention clips 49 in the shuttle 44. These components are hidden in FIG. 5 and are out of plane with the rest of the mechanism. Functionality of clips 49 may be similar or identical to the function of the retention clips 28 in device 10 shown in FIGS. 1-4.

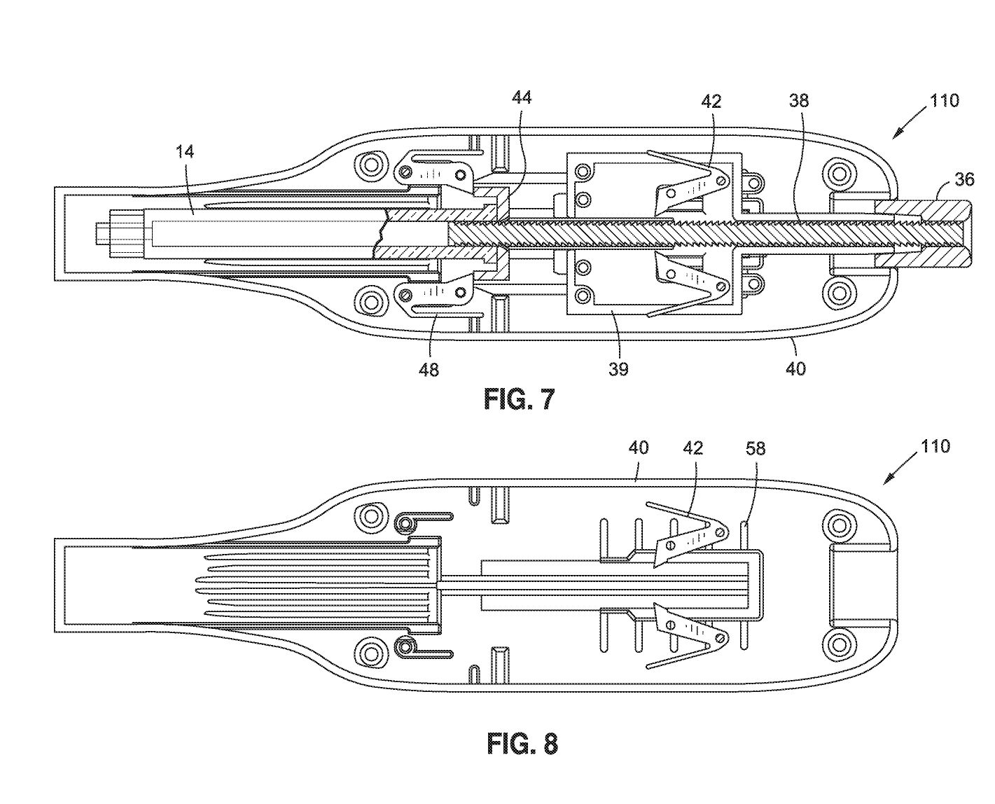

In the rest position shown in FIG. 7, the button/drive mechanism subassembly 39 is pulled back in the device 110 (toward the right) by the extension springs 56 (not shown in FIG. 7, but shown in FIG. 5). The shuttle 44 is pressed forward by the compression spring (not shown in FIG. 7) between it and the button/drive mechanism subassembly 39, but held back by the shuttle retention clips 49 (FIG. 6). As shown, driving clips 42 are in a compressed position, held away from the drive rack 38 by dowel pins that extend through the hole near the engagement point. FIG. 8 shows the ribs 58 of the housing 40 which interact with these dowel pins and hold the driving clips 42 in a compressed position.

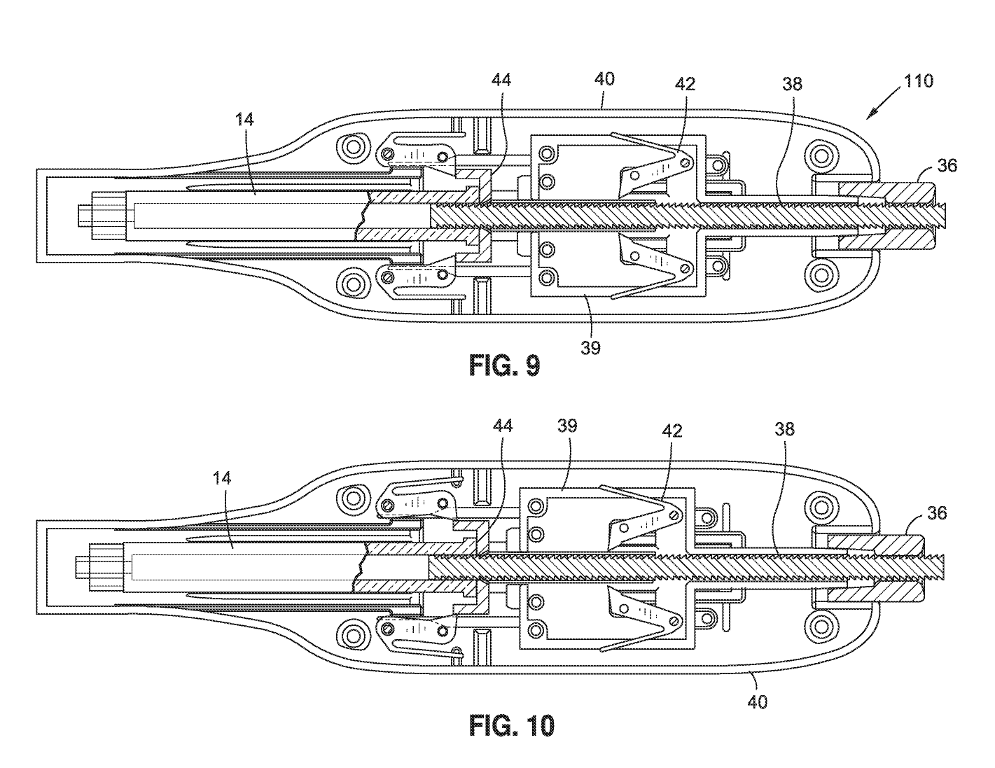

As shown in FIG. 9, as the button/drive mechanism 39 is actuated, the extension springs 56 extend and the compression spring 52 is compressed (since the shuttle 44 is held in place by the shuttle retention clips 49. Note that as the button/drive mechanism 39 moves forward, a feature on the forward (left side) legs begins to drive the shuttle retention clips outward. The shuttle retention clips have dowel pins at the pivot point at well as near the interface with the shuttle.

As shown in FIG. 10, the dowel pins near the interface with the shuttle extend such that as the button/drive mechanism legs move past, the shuttle retention clips begin to unlatch from the shuttle.

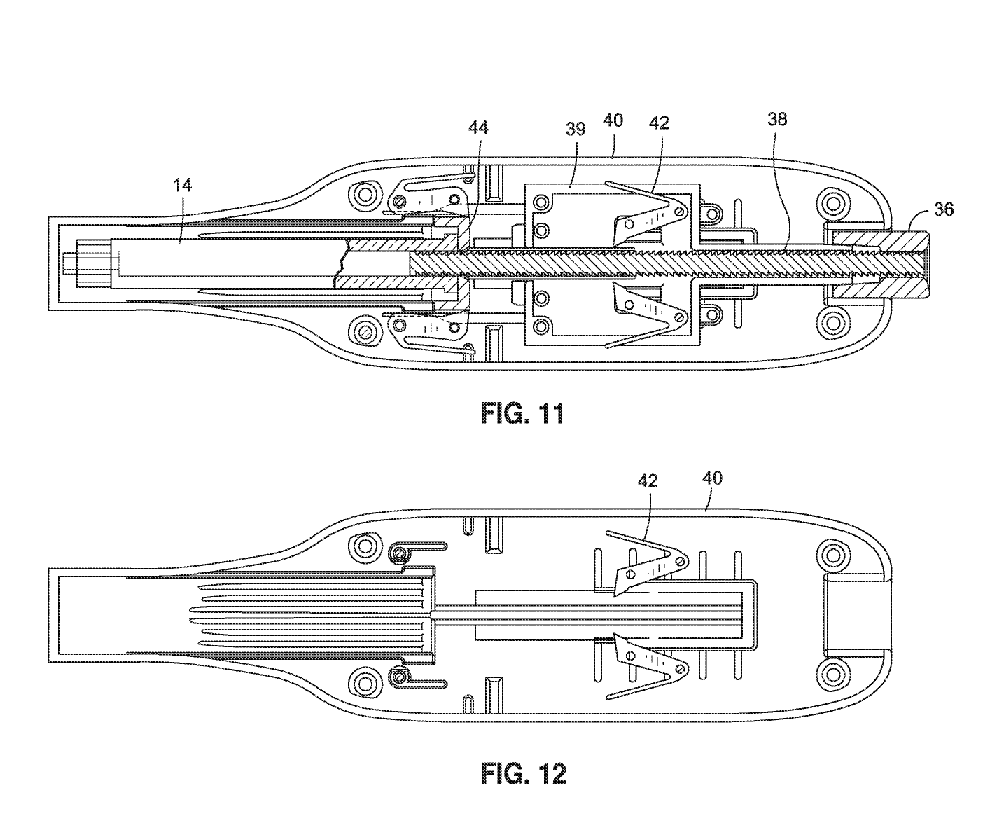

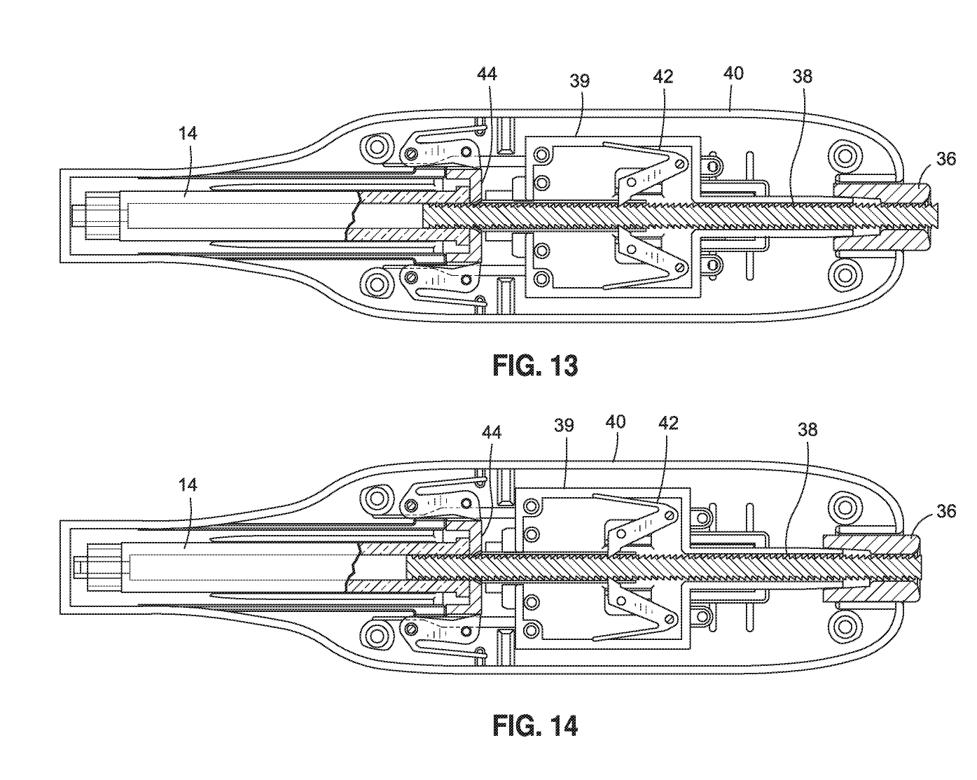

FIG. 11 shows that after the clips 42 unlatch, the shuttle snaps forward (to the left), driven by the compression spring between it and the button/drive mechanism. Throughout this portion of the stroke, the driving clips are still held away from the rack. FIG. 12 illustrates the position of the clips 42 at the time the needle(s) will have punctured the skin, during the snapping of the shuttle and syringe forward. At this point in the stroke, the dowel pins in the driving clips are close to a ramp in the rib that is holding them back. FIG. 13 shows the button/drive mechanism has moved forward, the dowel pins move down the ramp, and the clips are allowed to interface with the drive rack. FIG. 14 shows as the stroke continues, a dose is administered until a hard stop in reached in the system, indicating end of dose.

Once the button/drive mechanism is released, the system resets. The extension springs pull the button/drive mechanism toward the back of the housing to its initial position. As the button/drive mechanism moves back, the driving clips are reset into a position that is disengaged from the drive rack. The drive rack is held in place relative to the shuttle and syringe by the retention clips (not shown). The shuttle is captured by the button/drive mechanism, so as the button drive mechanism resets into its initial position, it pulls the shuttle into position. As the shuttle nears its initial position, the shuttle retention clips slip back into place and hold the shuttle back.

Although the invention has been described and illustrated with a certain degree of particularity, it is understood that the present disclosure has been made only by way of example, and that numerous changes in the combination and arrangement of parts can be resorted to by those skilled in the art without departing from the scope of the invention, as hereinafter claimed.

What is claimed is:

1. A device for delivering repeated doses of a composition into skin, the device comprising:
a handpiece comprising a main housing having a housing distal end;
a cartridge suitable for containing a composition and having a cartridge distal end configured to be coupled to a needle;
a plunger movable within the cartridge, and having a distal portion defining a head and a proximal portion defining a drive rack;
a shuttle, movable within the main housing and structured to receive the cartridge; and
retention clips for holding the shuttle forward in the housing and drive clips engaging the drive rack;
the device being structured and configured to allow one-way, incremental movement of the plunger each time the device is device actuated, thereby allowing delivery of repeated doses of the dermal filler into skin.

2. The device of claim 1 wherein the device is structured to be actuated upon pressing the device against the skin.

3. The device of claim 1 wherein the device further comprises a trigger which actuates the device.

4. The device of claim 1 wherein the drive rack comprises spaced apart teeth engaging the drive clips.

5. The device of claim 4 wherein the incremental movement of the plunger is defined by spacing between adjacent teeth.

6. A device for delivering repeated doses of a dermal filler superficially into skin, the device comprising:
a handpiece comprising a main housing having a housing distal end;
a cartridge suitable for containing a dermal filler and having a cartridge distal end configured to be coupled to a needle;
a plunger movable within the cartridge, and having a distal portion defining a head and a proximal portion defining a drive rack;
a shuttle, movable within the main housing and structured to receive the cartridge; and
retention clips for holding the shuttle forward in the housing and drive clips engaging the drive rack;
the device being structured and configured to allow one-way, incremental movement of the plunger each time the device is device actuated, thereby allowing delivery of repeated doses of the dermal filler into skin.

7. The device of claim 6 wherein the device is structured to be actuated upon pressing the device against the skin.

8. The device of claim 6 wherein the device further comprises a trigger which actuates the device.

9. The device of claim 6 wherein the drive rack comprises spaced apart teeth engaging the drive clips.

10. The device of claim 9 wherein the incremental movement of the plunger is defined by spacing between adjacent teeth.
